SEQUENCE LISTING (5 sequences)

SEQ ID NO: 1 - DNA, 48 bp, Artificial sequence
  Primer
nnnnnnggatccaagatggttgtcgtgcaagatggatcagtagccacc

SEQ ID NO: 2 - DNA, 42 bp, Artificial sequence
  Primer
nnnnnngtcgactcactgatcaagacttaggtctccccattg

SEQ ID NO: 3 - DNA, 33 bp, Artificial sequence
  Primer
nnnccatggttgtcgtgcaagatggatcagtag

SEQ ID NO: 4 - DNA, 34 bp, Artificial sequence
  Primer
caccgtcgacaatatgaaggctataaggatgacc

SEQ ID NO: 5 - DNA, 34 bp, Artificial sequence
  Primer
ggatcctatatgacaggtctaattcactttgaac

(19) United States
(12) Patent Application Publication
(10) Pub. No.: US 2011/0225663 A1
(43) Pub. Date: Sep. 15, 2011

(54) METHOD FOR PRODUCING EUKARYOTIC ORGANISMS WITH ENHANCED PATHOGEN RESISTANCE AND/OR RESISTANCE TO STRESS AND EUKARYOTIC TRANSGENIC ORGANISMS WITH ENHANCED PATHOGEN RESISTANCE AND/OR RESISTANCE TO STRESS

(75) Inventors: Antje Von Schaewen, Muenster (DE); Judith Scharte, Muenster (DE); Zeina Tjaden, Oldenburg (DE); Hardy Schoen, Berlin (DE); Olessja Becker, Muenster (DE); Kerstin Fischer, Muenster (DE)

(73) Assignee: WESTFAELISCHE WILHELMS-UNIVERSITAET MUENSTER, Muenster (DE)

(21) Appl. No.: 13/062,231

(22) PCT Filed: Sep. 4, 2009
(86) PCT No.: PCT/EP2009/006441

§ 371 Date: May 10, 2011

(30) Foreign Application Priority Data
Sep. 6, 2008 (EP) 08 015 750.6

(51) Int. Cl. A01K 67/033; A01H 5/00; C12N 1/00; A01H 1/00; C12N 15/74

(52) U.S. Cl. 800/13; 800/301; 435/254.11; 800/279; 435/471

(57) ABSTRACT

A method for producing an eukaryotic organism having at least one of enhanced pathogen resistance and resistance to stress includes expressing in a cytosol of the eukaryotic organism a glucose-6-phosphate dehydrogenase with an increased NADPH tolerance compared to an endogenous cytosolic glucose-6-phosphate dehydrogenase and at least one of reducing, eliminating and suppressing an activity of the endogenous cytosolic glucose-6-phosphate dehydrogenase.

### CROSS REFERENCE TO PRIOR APPLICATIONS

This application is a U.S National Phase application under 35 U.S.C. §371 of International Application No. PCT/EP2009/006441, filed on Sep. 4, 2009 and which claims benefit to European Patent Application No. 08 015 750.6, filed on Sep. 6, 2008. The International Application was published in English on Mar. 11, 2010 as WO 2010/025936 A1 under PCT Article 21(2).

### FIELD

The present invention relates to a method for producing eukaryotic organisms having enhanced pathogen resistance and/or resistance to stress, as well as to respective eukaryotic transgenic organisms showing enhanced pathogen resistance and/or resistance to stress.

### BACKGROUND

Organisms use a wide range of mechanisms to resist pathogens and stress, which are usually complicated and difficult to control. However, as organisms with enhanced resistance are desirable, for example, in the fields of agricultural crops, useful plants or domestic animals, a method for enhancing pathogen resistance and/or resistance to stress would be of great economic interest.

Glucose-6-phosphate dehydrogenase (G6PDH, EC 1.1.1.49) catalyses the first committed step of the oxidative pentose phosphate pathway (OPPP), an important catabolic route for the provision of NADPH and sugar phosphates. G6PDH is present in every eukaryotic organism and is normally contained in the cytosol as well as in various organelles. In plants, G6PDH activity is present at least in both cytosol and plastids, and possibly also in peroxisomes. The chloroplastic enzyme is known to be reductively inactivated in light by the ferredoxin/thioredoxin system to avoid futile interactions with the Calvin cycle during photosynthesis. The main role of G6PDH in plastids during the night or in heterotrophic tissues is the supply of reducing equivalents in the form of NADPH required for multiple anabolic reactions (such as amino acid or fatty acid synthesis). Moreover, sugar-phosphate intermediates that serve as precursors of nucleotides and secondary plant products are generated.

As mentioned above, enzyme reactions of the oxidative pentose phosphate pathway (OPPP) provide reduction power for anabolic biosyntheses in the form of NADPH. This also plays a role during early defence reactions, for example, upon elicitation of NADPH oxidase at the plasma membrane (“oxidative burst”), an early and evolutionary conserved attempt of eukaryotic cells to interfere with pathogen invasion (see FIGS. 1 and 2).

### SUMMARY

An aspect of the present invention is to provide eukaryotic organisms having enhanced fitness such as enhanced pathogen resistance and/or resistance to stress.

In an embodiment, the present invention provides a method for producing an eukaryotic organism having at least one of enhanced pathogen resistance and resistance to stress which includes expressing in a cytosol of the eukaryotic organism a glucose-6-phosphate dehydrogenase with an increased NADPH tolerance compared to an endogenous cytosolic glucose-6-phosphate dehydrogenase and at least one of reducing, eliminating and suppressing an activity of the endogenous cytosolic glucose-6-phosphate dehydrogenase.

### BRIEF DESCRIPTION OF THE DRAWINGS

The present invention is described in greater detail below on the basis of embodiments and of the drawings in which:

FIG. 1 shows a scheme of the G6PDH “enzyme replacement” strategy;

FIG. 2 shows G6PDH and NADPH oxidase inhibitors interfere with necrosis formation;

FIG. 3 shows construct design for the conducted studies;

FIG. 4 shows disease evaluation of parental cP2 lines versus their corresponding super-transformed RNAi progeny after pathogen challenge with P. nicotianae; and

FIG. 5 shows tobacco 1000-grain weights (mg) of different seed batches (n>5).

### SEQUENCE LISTING

The Sequence Listing associated with this application is filed in electronic form via EFS-Web and is hereby incorporated by reference into this specification in its entirety. The name of the text file containing the Sequence Listing is 10_Sequence_Listing. The size of the text file is 4,096 Bytes, and the text file was created on Mar. 4, 2011.

### DETAILED DESCRIPTION

In an embodiment, the present invention provides a method for producing eukaryotic organisms having enhanced pathogen resistance and/or resistance to stress, comprising the steps of:

expressing in the cytosol of said organism a glucose-6-phosphate dehydrogenase with increased NADPH tolerance compared to the endogenous cytosolic glucose-6-phosphate dehydrogenase, and

reducing, eliminating or suppressing the activity of the endogenous cytosolic glucose-6-phosphate dehydrogenase.

In this context, increased NADPH tolerance means that the K_{i[NADPH]} of the expressed G6PDH is enhanced compared to the endogenous cytosolic G6PDH. Furthermore, stress means abiotic as well as biotic stress and shall include every kind of stress which is always characterized by a primary oxidative burst at the plasma membrane. In the present invention, resistance to stress shall also include stress tolerance.

The present inventors have surprisingly found that when they introduced and expressed a glucose-6-phosphate dehydrogenase having increased NADPH tolerance in the cytosol of, for example, transgenic plants (a so-called ectopic expression), these organisms showed enhanced pathogen resistance as well as resistance to other stresses. For example, the replacement of cytosolic G6PDH by a kinetically superior isoenzyme was able to enhance resistance in the progeny of a susceptible plant variety.

While overexpression of G6PDH in chloroplasts or the expression of hundreds of different enzymes including G6PDH in various organisms is generally described in the prior art (see, for example, Debnam et al., Plant Journal, vol. 38, no. 1, pages 49-59 (2004), and WO 03/000898 A or WO 02/16655 A), the expression of a G6PDH having increased NADPH tolerance in the cytosol of an eukaryotic organism with a concurrent reduction, elimination or suppression of the activity of the endogenous cytosolic G6PDH has not been published before the priority date of the present invention.

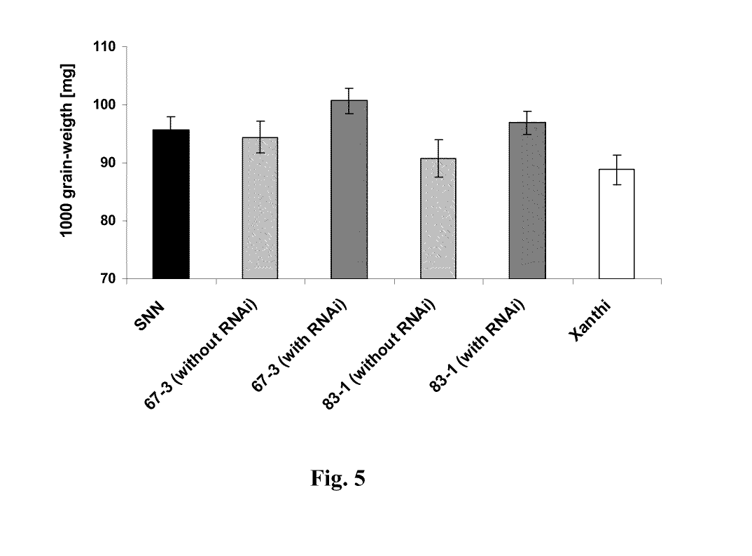

The present inventors also surprisingly found that the inventive organisms with a replacement of cytosolic G6PDH by a kinetically superior isoenzyme showed an increased harvest yield as shown by increased 1000-grain weights (see FIG. 5 and Table 2).

In addition to producing complete organisms, the inventive method may also be used to produce only parts of an organism, such as organs, tissues or cells. Therefore, for the purpose of the present application, the term “organism” is meant to also include parts of an organism as mentioned above. While complete organisms are nonhuman organisms, the parts of an organism also include human parts. Parts of an organism can, for example, be cells.

The G6PDH with increased NADPH tolerance can, for example, be an exogenous G6PDH, such as an isoenzyme from a different organism. If available, however, it is also possible to introduce and express a non-cytosolic G6PDH with increased NADPH tolerance from the same organism. The G6PDH with increased NADPH tolerance may furthermore be a mutated natural, an artificial and/or a genetically engineered modified enzyme.

In this regard, the present inventors found that the improvement in pathogen resistance and resistance to stress increased when the phylogenetic relationship was more distant (Solanaceae versus Arabidopsis G6PDH: 75% identity at the amino acid level).

In an embodiment of the present invention, the kinetic data of the G6PDH with increased NADPH tolerance fulfil the following relationship: K_{i[NADPH]}>K_{m[NADP+]}, for example, K_{i[NADPH]}≧2×K_{m[NADP+]}, for example, K_{i[NADPH]}≧5×K_{m[NADP+]} or, for example, K_{i[NADPH]}≧10×K_{m[NADP+]}. In this regard, a genetically engineered enzyme fulfilling the relationship K_{i[NADPH]}≧10×K_{m[NADP+]}, can, for example, be used.

In the context of the present specification, K_{i[NADPH]} and K_{m[NADP+]} are empirically determined kinetic enzyme parameters. Methods for determining these parameters are standard methods known to a person skilled in the art. As an example, these parameters may be determined as described in Wendt U K, Wenderoth I, Tegeler A, von Schaewen A: Molecular characterization of a novel glucose-6-phosphate dehydrogenase from potato (Solanum tuberosum L.), Plant J. 23, pp 723-733 (2000), for potato G6PDH, and in Wakao S and Benning C: Genome-wide analysis of glucose-6-phosphate dehydrogenases in Arabidopsis, Plant J. 41, pp 243-256 (2005), for Arabidopsis isoenzymes.

The G6PDH with increased NADPH tolerance can, for example, be an extracytoplasmic G6PDH (such as, for example, plastidic or peroxisomal) that naturally shows these kinetic properties. Examples include one of the two plastidic P2 isoenzymes (and possibly also the P1 isoform) derived from Arabidopsis, which is described in more detail below.

In an embodiment of the present invention, the G6PDH with increased NADPH tolerance can, for example, be overexpressed to reach a concentration in the cytosol of at least twofold (in relation to the concentration of the endogenous enzyme).

With regard to the step of reducing, eliminating or suppressing the activity of the endogenous cytosolic G6PDH, the endogenous cytosolic G6PDH can, for example, be replaced by an isoenzyme with increased NADPH tolerance in such a way that substantially no activity, or, for example, no activity of the endogenous cytosolic G6PDH remains.

Reducing, eliminating or suppressing the activity of endogenous cytosolic G6PDH can, for example, be carried out by targeted knock-out mutation or gene silencing, co-suppression, antisense or RNA interference, which are all standard methods belonging to the general knowledge of a person skilled in the art.

The eukaryotic organism can, for example, be selected from plants, animals or fungi, wherein the term “animals” includes vertebrates as well as invertebrates, and the term “plants” includes monocotyledons (monocots) as well as dicotyledons (dicots). As already mentioned above, the term “organism” also includes parts of an organism, such as human cell lines.

Since all kinds of stress are characterized by an oxidative burst, and this mechanism is present in every eukaryotic organism, it is considered that the present invention is in fact applicable to all kinds of eukaryotic organisms like plants, animals or fungi.

In case that the eukaryotic organism is a plant, such as a higher plant species, this plant can, for example, be selected from the group consisting of Solanaceae, soy bean, maize, rice, wheat, barley, rye, sugar cane, canola (rapeseed), cotton, sugar beet, switchgrass, Arabidopsis or else, for example, a plant selected from tobacco, tomato, potato, pepper or else. Grain crops can, for example, be used.

The present invention also relates to eukaryotic transgenic organisms having enhanced pathogen resistance and/or resistance to stress, characterized in that they express in their cytosol a G6PDH with increased NADPH tolerance compared to the endogenous cytosolic enzyme, while the activity of its endogenous cytosolic glucose-6-phosphate dehydrogenase is reduced, eliminated or suppressed.

These eukaryotic transgenic organisms are obtainable by the inventive method as described above, and they can, for example, be characterized by one or more of the features described above with respect to the inventive method.

The present invention also relates to the use of the inventive method described above for increasing the harvest yield of the respective eukaryotic organism.

In an embodiment, the present invention describes the benefits of enzyme replacement in the cytosol using an antisense approach for eliminating endogenous glucose-6-phosphate dehydrogenase activity combined with expression of an N-terminally truncated plastidial isoenzyme of P2 class, with enhanced K_{i[NADPH]}.

The present invention will now be described and explained with respect to the included figures and the following examples. These examples, however, are not intended to limit the scope of the present invention.

The figures show:

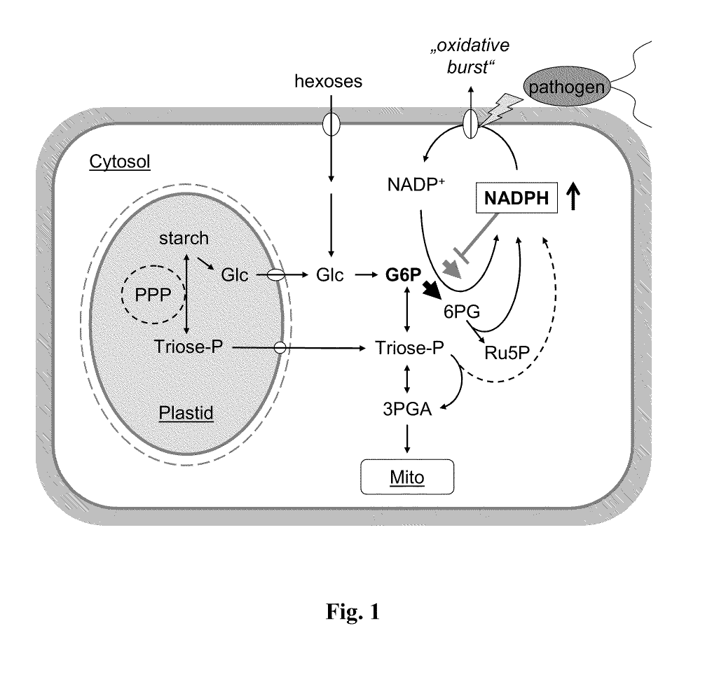

FIG. 1: Scheme of the G6PDH “enzyme replacement” strategy. Abbr.: Glc, glucose; G6P, glucose-6-phosphate; Mito, Mitochondrium; NADP⁺/H, nicotinamide-dinucleotide phosphate, oxidized or reduced form; 3PGA, 3-phosphoglycerate; 6PG, 6-phosphogluconate; PPP, pentose-phosphate pathway; Ru5P, ribulose-5-phosphate; Triose-P, triose phosphates DHAP and GA3P (dihydroxyacetone phosphate and glyceraldehyde-3-phosphate). For further explanation, see the text above.

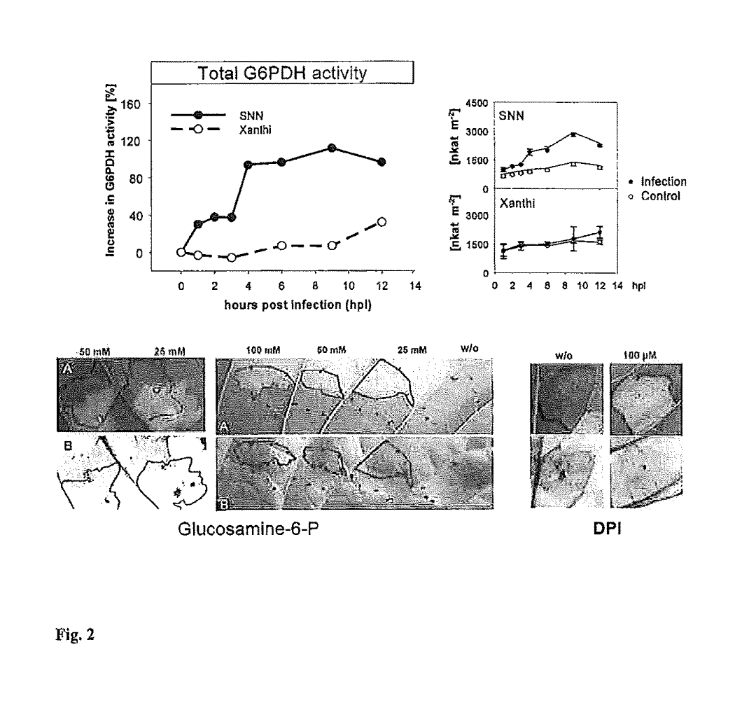

FIG. 2: G6PDH and NADPH oxidase inhibitors interfere with necrosis formation. Top, G6PDH activity increases dramatically in the resistant tobacco wild type variety SNN after infection with Phythophthora nicotianae (P. nicotianae; see also Scharte J, Schön H, Weis E: Photosynthesis and carbohydrate metabolism in tobacco leaves during an incompatible interaction with Phytophthora nicotianae, Plant, Cell Environ. 28, pp 1421-1435 (2005)) compared to susceptible wild type Xanthi plants. This suggested a key role for G6PDH in successful plant defence. Bottom, Interference of Glucosamin-6-P (G6PDH inhibitor, infiltrated area marked red) and Diphenylene iodonium (DPI, NADPH oxidase inhibitor) with defence-induced H₂O₂ production (visualized by DAB-staining—after removal of chlorophyll) and the formation of hypersensitive lesions indicate that efficient plant defence depends upon NADPH availability in the cytosol, stemming mostly from the OPPP. Abbr.: w/o, without.

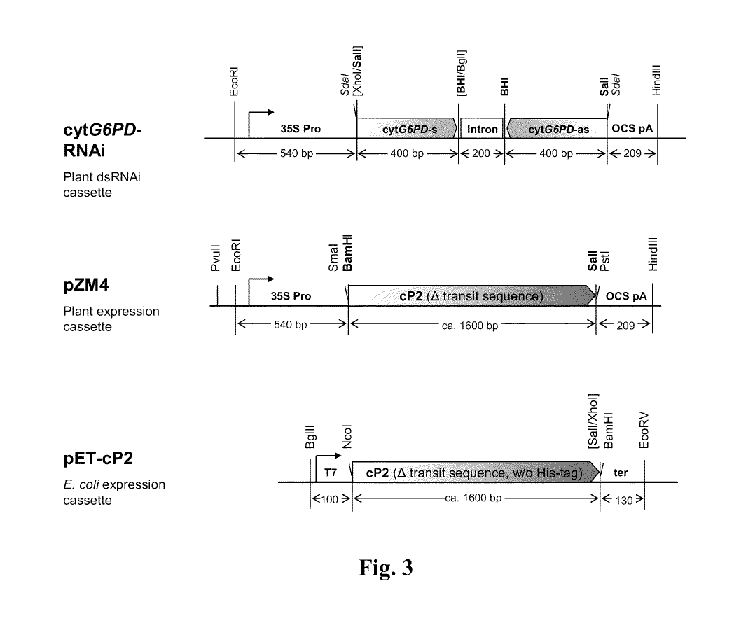

FIG. 3: Construct design for the conducted studies.

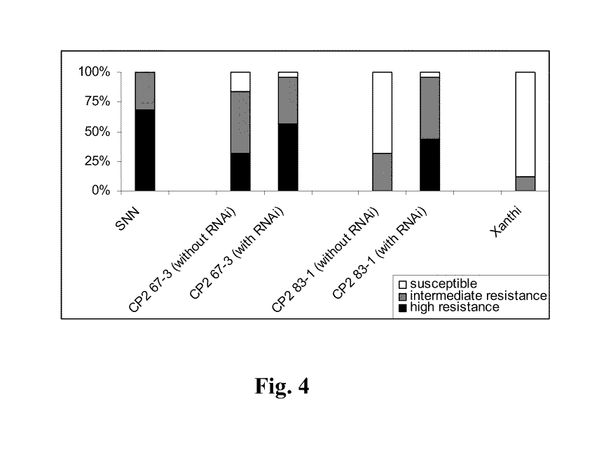

FIG. 4: Disease evaluation of parental cP2 lines versus their corresponding super-transformed RNAi progeny after pathogen challenge with P. nicotianae.

Side-by side analysis of weak and strong parental cP2 lines versus their corresponding super-transformed RNAi progeny compared to SNN and Xanthi wild type varieties.

The extent of hypersensitive lesions formed at fifty infiltration sites with zoospores of P. nicotianae on 5 plants (three or more independent rounds of infection) was evaluated after 2 days: High resistance, 80-100% lpa (lesions per area); intermediate resistance, 35-80% lpa; susceptible response, 0-10% lpa at infiltration site. The data obtained and illustrated in FIG. 4 are also summarized in Table 1 below:

TABLE 1

| | | Intermediate | |
|---|---|---|---|
| Variety/trangenic line | Susceptible | resistance | High resistance |
| SNN (resistant wt) | 0 | 16 | 34 |
| 67-3 (without RNAi) | 8 | 26 | 16 |
| 67-3 (with RNAi) | 2 | 20 | 28 |
| 83-1 (without RNAi) | 34 | 16 | 0 |
| 83-1 (with RNAi) | 2 | 26 | 22 |
| Xanthi (susceptible wt) | 44 | 6 | 0 |

FIG. 5: Tobacco 1000-grain weights (mg) of different seed batches (n>5) were determined upon collection in pre-weighed Eppendorf tubes using an analytical balance.

Mean values, standard deviations (SD) and standard errors (SE) were calculated with Excel 2003 (v11.0, MICROSOFT, Redmond, USA). Differences described as significant were calculated by the t-test algorithm incorporated into Microsoft Excel. Data are shown as mean mg±SE of at least five individual seed batches. Asterisks indicate significant differences compared to Xanthi wild type as determined by the student's t-test (*, p<0.05, **; p<0.01; ***, p<0.001). The data obtained and illustrated in FIG. 5 are also summarized in Table 2 below:

TABLE 2

| | | 67-3 | 67-3 | 83-1 | 83-1 | |
|---|---|---|---|---|---|---|
| | | (without | (with | (without | (with | |
| | SNN | RNAi) | RNAi) | RNAi) | RNAi) | Xanthi |
| Mean | 95.7 | 94.4 | 100.7 | 90.8 | 97 | 88.8 |
| SD | 5.9 | 8.6 | 5.2 | 6.5 | 5.9 | 7.1 |
| SE | 2.5 | 2.7 | 2.1 | 3.2 | 2 | 2.5 |
| t-test | | 0.321 | 0.003 | 0.456 | 0.033 | |

SNN = resistant,
Xanthi = susceptible Nicotiana tabacum variety.

### EXAMPLES

Tobacco Nicotiana tabacum lines of the susceptible variety Xanthi (as described in Way H M, Kazan K, Mitter N, Goulter K G, Birch R G, Manners J M: Expression of the ShPAL phenylalanine ammonia lyase gene of Stylosanthes humilis in transgenic tobacco leads to enhanced disease resistance but impaired plant growth, Physiol Mol Plant Pathol 60, pp 275-282 (2002)) were engineered to replace endogenous cytsolic G6PDH activity by an N-terminally truncated plastidial isoenzyme with superior biochemical characteristics (see FIG. 3). Transgenic lines expressing Arabidopsis thaliana G6PD isoform At1g24280 (P2 class) without transit peptide were additionally transformed with an RNAi construct eliminating expression of endogenous tobacco G6PD isoforms in the cytosol. “Enzyme replaced” Xanthi super-transformants reacted strongly (hypersensitive) after zoospore infection with Phytophthora nicotianae comparable to the natural tobacco wild type variety Samsun N N. Moreover, extent of necrosis formation was independent of the response displayed by the parental lines (weak to intermediate responses, see FIG. 4). This is an example that effective metabolic channelling ensues only when competing enzyme activities are eliminated in the same cellular compartment.

### Example 1

### Measurement of G6PDH and Specific Inhibitor Studies

### Photometric Determination of G6PDH Activity

Freshly cut leaf discs were frozen in liquid N₂ and ground to a fine powder. Extraction and determination of G6PDH activity was according to the method described by Fickenscher K and Scheibe R: Purification and properties of the cytoplasmatic glucose-6-P dehydrogenase from pea leaves, Arch Biochem Biophys 247, pp 393-402 (1986).

### Inhibitor Studies

Evidence for the involvement of NADPH oxidase as the source of ROS-formation is provided by the inhibitory effect of the flavoprotein inhibitor diphenylene iodonium (DPI). DPI is a well-known inhibitor of the mammalian neutrophil oxidase and also inhibits plant NAD(P)H oxidases (as described in Pugin A, Frachisse J M, Tavernier E, Bligny R, Gout E, Douce R, Guern J: Early events induced by the elicitor Cryptogein in tobacco cells: Involvement of a plasma membrane NADPH Oxidase and activation of glycolysis and the pentose phosphate pathway, Plant Cell 9, pp 2077-2091 (1997) and references cited therein). NADPH oxidase was shown to be the main source for extracellular ROS production (oxidative burst) in plants upon elicitation (as described in Pugin et al., 1997, as referenced above). This leads to the formation of hypersensitive lesions in leaf tissue of resistant varieties after pathogen infection (incompatible interaction).

When tobacco leaves were treated with 25-100 mM DPI, infection with P. nicotianae did not induce the formation of hypersensitive lesions.

The involvement of the oxidative pentose phosphate pathway (OPPP) in the oxidative burst of plants was shown by inhibitor studies with Glucosamine-6 phosphate (Glucosamine-6-P). This is a well-known competitive inhibitor for G6PDH (as described in Glaser B L and Brown D H: Purification and properties of D-glucose 6-phosphate dehydrogenase, J Biol Chem 216, pp 67-79 (1955)), the first enzyme of the OPPP, which transforms G6P into 6-phosphogluconolactone. When tobacco leaves are treated with 25-50 mM GN6P, infection with P. nicotianae did not result in detectable ROS production.

### Example 2

### Zoospore Infiltration, H₂O₂ Detection, and Evaluation of Leaf Necroses Formation

### Oomycete Growth, Zoospore Production, and Inoculation

Phytophthora nicotianae van Breda de Haan isolate 1828 (DSMZ, Braunschweig, GER) was cultivated at 24° C. on clarified tomato agar as described by von Broembsen S L and Deacon J W: Germination and further zoospore release from zoospore cysts of Phytophthora parasitica, Mycol Res 100, pp 1498-1504 (1996). Zoospores were produced under aseptic conditions as described by von Broembsen von Broembsen S L and Deacon J W: Germination and further zoospore release from zoospore cysts of Phytophthora parasitica, Mycol Res 100, pp 1498-1504 (1996). Source leaves from 8- to 10-week-old tobacco plants were infiltrated with a suspension containing 500 to 1,000 zoospores μL⁻¹ as described by Colas V, Conrod S, Venard P, Keller H, Ricci P, Panabieres F: Elicitin genes expressed in vitro by certain tobacco isolates of Phytophthora parasitica are down regulated during compatible interactions, Mol Plant Microbe Interact 14, pp 326-335 (2001). This zoospore-leaf infiltration assay was chosen to achieve a rapid, synchronized infection start in all parenchymatic cells of the infiltrated leaf area. For mock-inoculation, sterile tap water was infiltrated, and is further referred to as control. To take into account individual plant or developmental variations, samples from control and infection sites were excised from adjacent intercostal areas of the same source leaf. Plant inoculation was always performed at the beginning of the photoperiod.

### Histochemical Detection of H₂O₂

Hydrogen peroxide (H₂O₂) accumulation was detected by in situ staining with 3,3-diaminobenzidine (DAB) following a modified protocol of Thordal-Christensen H, Zhang Z G, Wei Y D, Collinge D B: Subcellular localization of H₂O₂ in plants: H₂O₂ accumulation in papillae and hypersensitive response during the barley-powdery mildew interaction, Plant J 11, pp 1187-1194 (1997). Leaves were placed in DAB solution (1 mg mL⁻¹, pH 3.8) for 6 h. A dark-brown polymerization product formed at sites where DAB reacts with H₂O₂ produced by the tissue. Incubations were stopped and leaf tissue was simultaneously cleared from chlorophyll by boiling in ethanol for 10 min.

### Determination of the Extent of Hypersensitive Lesions

The extent of hypersensitive lesions formed at fifty infiltration sites with zoospores of Phytophthora nicotianae on 5 plants (three or more independent rounds of infections) were evaluated after 2 days: High resistance, 80-100% lpa (lesions per area); intermediate resistance, 35-80% lpa; susceptible response, 0-10% lpa at infiltration site.

### Example 3

### Cloning Strategy of cP2 Plant Expression Constructs

Total RNA was isolated from Arabidopsis leaves (as described in Logemann J, Schell J, Willmitzer L: Improved method for the isolation of RNA from plant tissues, Anal Biochem 163, pp 16-20 (1987)) and reverse transcribed from mRNA using a polyA primer mix (5′-A₃₀-C/G/T-3′) and Superscript II (Invitrogen) according to a protocol of the supplier. Truncated Arabidopsis P2 cDNA fragments (At1g24280, 5′ delta 195 bp termed cP2) were amplified from 1^{st} strand cDNA using primers ZM_S2 and ZM_S3 and PfuI DNA polymerase (Stratagene), and directly cloned into BamHI and SalI opened pBluescript SK vector (Stratagene) yielding pZM3. Similarly, BamHI and SalI digested cP2 fragments were introduced into the multiple cloning site of pA35 (plant expression cassette assembled in pUC18, as described in Höfte H, Faye L, Dickinson C, Herman E M, Chrispeels M J: The protein-body proteins phytohemagglutinin and tonoplast intrinsic protein are targeted to vacuoles in leaves of transgenic tobacco, Panta 184, pp 431-437 (1991)) between CaMV 35S promoter and OCS polyadenylation signal yielding pZM4. The entire plant expression cassette was transferred by complete HindIII and partial PvuII digest into HindIII and SnaBI opened vector pGSC1704 [HygR] (Plant Genetic Systems), yielding final binary construct pZM5 suited for Agrobacterium-mediated stable plant transformation (see Example 5 below).

Primers for Amplification of Arabidopsis cP2 cDNA Fragments:

ZM_S2 sense (BamHI recognition site underlined,
start codon bold)
(SEQ ID NO: 1)
5′-N₆-GGA TCC AAG ATG GTT GTC GTG CAA GAT GGA
TCA GTA GCC ACC-3′
ZM_A3 antisense (SalI recognition site
underlined, stop codon bold)
(SEQ ID NO: 2)
5′-N₆-GTC GAC TCA CTG ATC AAG ACT TAG GTC TCC
CCA TTG-3′

### Example 4

### G6PDH Activity Test of the Recombinant cP2 Enzyme in E. Coli

For cloning into E. coli expression vector pET16b (Novagen) cP2 cDNA fragments were amplified from pZM4 (FIG. 3) using primers pET-cP2 sense and ZM_A3 (antisense, see above) and Phusion DNA polymerase (Finzymes). PCR products were digested with NcoI and SalI and cloned in E. coli XL1 blue (Stratagene) after ligation to NcoI-XhoI opened vector pET16b (Novagen), yielding pET-cP2. G6PDH activity of the cP2 enzyme was determined in a G6PDH-deficient E. coli strain. Host strain BL21(DE3) pLysS (Novagen) was modified by P1 transduction using E. coli zwf minus strain SU294 (as described by Lee W T and Levy H R: Lysine-21 of Leuconostoc mesenteroides glucose 6-phosphate dehydrogenase participates in substrate binding through charge-charge interaction, Protein Sci 1, pp 329-334 (1992)) resulting in BL21^{G6PDminus} zwf::Tn10[TetR] (Christian Schwöppe and Antje von Schaewen, unpublished). After retransformation, cP2 expression was induced in logarithmically growing BL21^{G6PDminus}: pET-cP2 cultures by adding IPTG (1 mM f.c) and allowed to grow for 2-3 h at 37° C. E. coli cells were harvested by centrifugation and adjusted to 10 OD₆₀₀. Extraction was in 100 mM NaH₂PO₄, 10 mM Tris-NaOH pH 8 supplemented with 0.1 mM Pefabloc SC and 0.02 mM NADP (to stabilize G6PDH) by 3 times sonication for 10 sec at 50 W (Branson sonifier). G6PDH activity of cP2 was characterized by K_{m[G6P]}=0.58 mM, and K_{i[NADPH]}=4.6 μM>K_{m[NADP*]}=2.4 μM. The latter values differ slightly from those previously described by Wakao S and Benning C: Genome-wide analysis of glucose-6-phosphate dehydrogenases in Arabidopsis, Plant J. 41, pp 243-256 (2005) for the recombinant enzyme with C-terminal Strep-tag (K_{i[NADPH]}=22 μM>K_{m[NADP+]}=17 μM).

Specific Primer for Cloning a pET16b-cP2 Expression Construct (Without His-Tag)

pET-cP2 sense (NcoI recognition site
underlined, start codon bold)
(SEQ ID NO: 3)
5′-NNNCC ATG GTT GTC GTG CAA GAT GGA TCA G
TA G-3′

### Example 5

### Generation of Tobacco Plants that Overexpress cP2 (At1g24280) in the Cytosol

Binary construct pZM5 (Example 4, see above) was directly transformed into Agrobacterium strain GV2260 (as described in Deblaere R, Bytebier B, De Greve H, Debroeck F, Schell J, van Montagu M, Leemans J: Efficient octopine Ti plasmid-derived vectors of Agrobacterium-mediated gene transfer to plants, Nucl Acids Res 13, pp 4777-4788 (1988)) according to a protocol described by Höfgen R, Willmitzer L: Storage of competent cells for Agrobacterium transformation, Nucl Acids Res 16, pg 9877 (1988). Generation of transgenic tobacco plants was by Agrobacterium cocultivation of Nicotiana tabacum var. Xanthi leaf discs with GV2260:pZM5 followed by a combined callus-shoot regeneration protocol as described by Voelker T, Sturm A, Chrispeels M J: Differences in expression between two seed lectin alleles obtained from normal and lectin-deficient beans are maintained in transgenic tobacco, EMBO J. 6, pp 3571-3577 (1987). Xanthi transformants were selected for high expression of cP2 in T0 using Northern-blot analyses, and by immunoblot analyses in T1 and all following generations (not shown) using a G6PDH antiserum specific for plant P2 isoenzymes (as described in Wendt U K, Wenderoth I, Tegeler A, von Schaewen A: Molecular characterization of a novel glucose-6-phosphate dehydrogenase from potato (Solanum tuberosum L.), Plant J. 23, pp 723-733 (2000)).

### Example 6

### Generation of Xanthi-cP2 Plants with Reduced Levels of Endogenous Cytosolic G6PDH Activity by Supertransformation with a cytG6PD-dsRNAi Construct

Cloning of a cytG6PD-dsRNAi Construct

The cytG6PD-dsRNAi construct was designed based on tobacco cytG6PD isoforms (not shown). Approximately 400 bp were amplified by RT-PCR from total leaf RNA isolated from the Nicotiana tabacum variety Xanthi. The resulting fragment was inserted twice into vector pUC-RNAi (as described in Chen S, Hofius D, Sonnewald U, Bornke F: Temporal and spatial control of gene silencing in transgenic plants by inducible expression of double-stranded RNA, Plant J. 36: pp 731-740 (2003)) flanking the central first intron of potato GA20-Oxidase. The first insertion was via SalI/BamHI and the second insertion via XhoI/BglII compatible ends.

Primers for Cloning a Tobacco G6PD-dsRNAi Construct in pUC-RNAi (As Described in Chen et al., 2003, as Defined Above)

cytG6PD-s (sense, SalI site underlined)
(SEQ ID NO: 4)
5′-CACCGTCGACAATATGAAGGCTATAAGGATGACC-3′
cytG6PD-as (antisense, BamHI site
underlined)
(SEQ ID NO: 5)
5′-GGATCCTATATGACAGGTCTAATTCACTTTGAAC-3′

The entire dsRNAi region was then released by restriction digest with PstI and the expression cassette was inserted (between the strong CaMV 35S promoter and OCS polyadenylation signal) of SdaI opened binary vector pBinAR[Kan] (as described in Höfgen R and Willmitzer L: Biochemical and genetic analysis of different patatin isoforms expressed in various organs of potato (Solanum tuberosum), Plant Sci 66, pp 221-230 (1990)).

Supertransformation of Xanthi-cP2 Lines Using the Binary cytG6PD-RNAi Construct

Leaf discs of two independent T1 Xanthi-cP2 lines (weak cP2 83-1 and strong cP2 67-3) were transformed by the leaf-disc method (as in Example 5 above) using Agrobacterium strain GV2260 carrying the pBinAR-cytG6PD-RNAi construct. Regeneration of supertransformants was on double selective media containing 100 mg/l Kanamycin and 40 mg/l Hygromycin B.

The present invention is not limited to embodiments described herein; reference should be had to the appended claims.

1-15. (canceled)

16. Method for producing an eukaryotic organism having at least one of enhanced pathogen resistance and resistance to stress, the method comprising:
expressing in a cytosol of the eukaryotic organism a glucose-6-phosphate dehydrogenase with an increased NADPH tolerance compared to an endogenous cytosolic glucose-6-phosphate dehydrogenase; and
at least one of reducing, eliminating and suppressing an activity of the endogenous cytosolic glucose-6-phosphate dehydrogenase.

17. The method as recited in claim 16, wherein the glucose-6-phosphate dehydrogenase with an increased NADPH tolerance is an exogenous glucose-6-phosphate dehydrogenase.

18. The method as recited in claim 16, wherein a kinetic data of the glucose-6-phosphate dehydrogenase with an increased NADPH tolerance fulfils at least one of the relationships K_{i[NADPH]}>K_{m[NADP+]}, K_{i[NADPH]}≧2×K_{m[NADP+]}, K_{i[NADPH]}≧5×K_{m[NADP+]} and K_{i[NADPH]}≧10×K_{m[NADP+]}.

19. The method as recited in claim 16, wherein the glucose-6-phosphate dehydrogenase with an increased NADPH tolerance is a plastidic or a peroxisomal glucose-6-phosphate dehydrogenase.

20. The method as recited in claim 16, wherein the glucose-6-phosphate dehydrogenase with an increased NADPH tolerance is overexpressed.

21. The method as recited in claim 20, wherein the glucose-6-phosphate dehydrogenase with an increased NADPH tolerance is overexpressed so as to reach a twofold or greater concentration in the cytosol.

22. The method as recited in claim 16, wherein the activity of the endogenous cytosolic glucose-6-phosphate dehydrogenase is at least one of reduced, eliminated and suppressed by at least one of a targeted knock-out mutation, a gene silencing, a co-suppression, an antisense and an RNA interference.

23. The method as recited in claim 16, wherein the eukaryotic organism is selected from at least one of a plant, an animal and a fungi.

24. The method as recited in claim 23, wherein the plant is selected from a higher plant species.

25. The method as recited in claim 23, wherein the plant is at least one of Solanaceae, soy bean, maize, rice, wheat, barley, rye, sugar cane, canola, cotton and Arabidopsis.

26. The method as recited in claim 23, wherein the plant is selected from at least one of tobacco, tomato, potato and pepper.

27. An eukaryotic transgenic organism having at least one of enhanced pathogen resistance and resistance to stress, wherein a cytosol of the eukaryotic transgenic organism expresses a glucose-6-phosphate dehydrogenase with an increased NADPH tolerance compared to an endogenous cytosolic glucose-6-phosphate dehydrogenase, and wherein an activity of the eukaryotic transgenic organism's endogenous cytosolic glucose-6-phosphate dehydrogenase is at least one of reduced, eliminated and suppressed.

28. The eukaryotic transgenic organism as recited in claim 27, wherein a kinetic data of the glucose-6-phosphate dehydrogenase with an increased NADPH tolerance fulfils at least one of the following relationships: K_{i[NADPH]}>K_{m[NADP+], K}_{i[NADPH]}≧2×K_{m[NADP+]}, K_{i[NADPH]}≧5×K_{m[NADP+]} and K_{i[NADPH]}≧10×K_{m[NADP+]}.

29. The eukaryotic transgenic organism as recited in claim 27, wherein the glucose-6-phosphate dehydrogenase with an increased NADPH tolerance is at least one of an exogenous glucose-6-phosphate dehydrogenase, a plastidic and a peroxisomal glucose-6-phosphate dehydrogenase.

30. The eukaryotic transgenic organism as recited in claim 27, wherein the eukaryotic transgenic organism is selected from at least one of a plant, an animal and a fungi.

31. The eukaryotic transgenic organism as recited in claim 30, wherein the plant is a higher plant species.

32. The eukaryotic transgenic organism as recited in claim 31, wherein the higher plant species is selected from at least one of Solanaceae, soy bean, maize, rice, wheat, barley, rye, sugar cane, canola, cotton and Arabidopsis, tobacco, tomato, potato and pepper.

33. Method of using an eukaryotic organism to increase a harvest yield, the method comprising:
providing an eukaryotic organism having at least one of enhanced pathogen resistance and resistance to stress as recited in claim 16; and
using the eukaryotic organism to increase a harvest yield.

34. Method of using as recited in claim 33, wherein the eukaryotic organism is a higher plant species.

35. The eukaryotic transgenic organism as recited in claim 34, wherein the higher plant species is selected from at least one of Solanaceae, soy bean, maize, rice, wheat, barley, rye, sugar cane, canola, cotton and Arabidopsis, tobacco, tomato, potato and pepper.
